(19) United States
(12) Patent Application Publication
(10) Pub. No.: US 2005/0244366 A1
(43) Pub. Date: Nov. 3, 2005

(54) Dressing and method of treatment for a wound

(76) Inventor: Lisa M. Brothers, Ingleside, IL (US)

Correspondence Address: Robert W. Diehl, Esq.;Wallenstein Wagner & Rockey, Ltd., 53rd Floor, 311 South Wacker Drive, Chicago, IL, 60606-6630, US

(21) Appl. No.: 10/837,453

(22) Filed: Apr. 30, 2004

(51) Int. Cl. A61K 31/785; A61K 9/00; A61L 15/00

(52) U.S. Cl. 424/78.27; 424/445

(57) ABSTRACT

A dressing for a wound site and method of using same. The dressing includes a first layer comprising a butyl-cyanoacrylate adhesive applied directly to the wound site and extending a distance beyond the wound site to form a peripheral edge. The dressing also includes a second layer comprising a butyl-cyanoacrylate adhesive applied directly to the first layer and overlapping the peripheral edge of the first layer a distance. In a moderate-tension wound the second layer extends the entire area of the first layer, but also extends a distance beyond the peripheral edge.

### RELATED APPLICATIONS

The present application is related to and claims priority of co-pending U.S. patent application Ser. No. ______ (Attorney Docket No. 3594 P002), filed concurrently herewith and hereby incorporated by reference.

### TECHNICAL FIELD

This invention relates generally to the treatment of a wound. More particularly, this invention relates to the treatment of a moderate-tension wounds and pediatric wounds via the application of a 1-butyl cyanoacrylate material directly to the wound site.

### BACKGROUND OF THE INVENTION

In the medical field, there are several different methods currently known for the treating and closing of wounds resulting from surgical incisions, lacerations, punctures and the like. Devices such as sutures, staples, skin tapes, and adhesives have been used when treating these different types of wounds. However, many of these devices have shortcomings. For example, sutures and surgical staples involve inflicting additional trauma to the wound surface since the needle or staple must pass through the tissue on the edges of the wound. Sutures and staples can also cause increased tension at the site, which will result in increased scarring. Further, when sutures and staples are used, the body treats them as foreign objects. This causes the body to increase its rate of reepithelialization at the wound site. The greater the rate of reepithelialization, the greater amount of scar tissue produced. Finally, sutures or surgical staples often require removal in a second procedure, resulting in additional inconveniences and potential discomfort to the patient.

Surgical strips, on the other hand, are typically used on superficial wounds due to their poor tensile strength. The low holding power of surgical strips causes the strips to prematurely fall off and the wound to open, particularly when in the presence of moisture. All of these examples hold true for wounds in human and veterinary patients.

For these reasons, it has become more common for medical personnel to apply adhesives directly to a wound. For example, 1-butyl cyanoacrylate, commonly sold under the names Indermil™, Histacryl Blue™, and Vetbond™ have been used to aid in the closure of wounds. After the wound has reached homeostasis and the wound edges approximated, these materials are applied directly to the wound, permitting the wound to heal over time without the use of sutures, staples, and skin tapes. However, the use of 1-butyl cyanoacrylate has been thus far fairly limited. Due to the known brittleness of the 1-butyl cyanoacrylate, the material has problems flexing with the movement of the body. This has led those skilled in the art to use 1-butyl cyanoacrylate material in only short, low-tension wounds. When applied properly, 1-butyl cyanoacrylate has the potential for much more than what it is presently used for. Butyls are cheaper, easier, and faster to use than octyl cyanoacrylates.

Another type of material that is used in the medical field is 2-octyl cyanoacrylate, which is sold under the commercial name Dermabond™ and Nexaband™. 2-octyl cyanoacrylate is a type of glue that can be used on any area of the body for wounds of varying lengths and sizes. Currently, the recommended and approved technique for applying 2-octyl cyanoacrylate to a wound is by applying multiple layers of 2-octyl cyanoacrylate over the top of the wound. U.S. Pat. No. 6,479,725 to Brothers describes a technique for dressing high-tension wounds using octyl cyanoacrylate. However, this technique has problems; the tensile strength of the layers is higher than surrounding skin. This condition may be exceptionally prevalent in patients with poor skin turgor, such as the elderly and those on extended regiments of steroids. These conditions cause dehiscence (i.e., breaking of the skin) at either edge of the dressing, thus creating additional wounds needing repair. The patient experiences additional inconveniences and discomfort because the wound has to be treated again. If the dehiscence happens more than 24 hours after an injury, all of the patient's wounds will have to be sutured due to the increased risk of infection.

Still another problem with prior art methods is the application to pediatric and mentally debilitated patients. Such patients will tend to pick at dressings and wounds, causing irritation, reopening, infection, or the like.

The present invention improves on current techniques by limiting the layers necessary, and thereby decreasing the tensile strength. By decreasing the tensile strength, a tensile strength is achieved that is closer to skin's own strength and avoiding dehiscence. The present invention discloses a new dressing having a much lower tensile strength that is very close to the actual strength of human skin and animal hide. By creating new techniques that utilize fewer layers, less adhesive material is used, resulting in cost savings. Instead of using one vial for every four inches, one vial may be used for 5 to 6 inches.

The 1-butyl cyanoacrylates have also been polymerized for the use of wound closure inside the body. The development of oxyalkene, alkylene carbonate, alkyl ester and alkyl cyanoacrylate, among others, have facilitated this. The same principles of application techniques are applicable to internal and external use in both humans and animal patients. This opens the doors for many different advances in medical care.

### SUMMARY OF THE INVENTION

A dressing and method for treating a wound on a pediatric patient or a patient having poor skin turgor is disclosed. One embodiment of the disclosed method comprises the steps of preparing the wound site for treatment, applying a first layer comprising a butyl-cyanacrylate adhesive directly to the wound site such that a peripheral edge of the first layer extends beyond all edges of the wound site, and applying a second layer comprising a butyl-cyanacrylate adhesive directly over the peripheral edge of the first layer and extending beyond all edges of the wound.

It is an aspect of one embodiment of the present invention that the wound dressing consist of a first layer of an adhesive which extends beyond the peripheral edge of the wound site, and a second layer of an adhesive which is applied to overlap the peripheral edge of the first layer a distance.

It is an aspect of an embodiment of the present invention that the overlapping distance is at least one-half millimeter (0.5 mm). It is another aspect of another embodiment that the overlapping distance is at least 100% of the first layer.

These and other objects, advantages and features of the invention, together with organization and manner of operation thereof, will become apparent from the following detailed description when taken into conjunction with the accompanying drawings, wherein like elements have like numerals throughout the drawings described below.

### BRIEF DESCRIPTION OF THE DRAWINGS

FIG. 1 is an isometric view of one embodiment of the present invention illustrating the application of cyanoacrylate to a wound site.

FIG. 2 is a top view of one embodiment of the present invention illustrating a wound after two layers of cyanoacrylate have been applied.

FIG. 3 is a top view of one embodiment of the present invention illustrating a wound after three layers of cyanoacrylate have been applied.

FIG. 4 is a top view of one embodiment of the present invention illustrating, for example, a pediatric wound after two layers of cyanoacrylate have been applied.

FIG. 5 is a top view of an alternative embodiment of the present invention illustrating, for example, a moderate-tension wound site after two layers of cyanoacrylate have been applied.

### DETAILED DESCRIPTION OF THE INVENTION

While the present invention is susceptible of embodiment in many different forms, this disclosure will describe in detail at least one preferred embodiment, and possible alternative embodiments, of the invention with the understanding that the present disclosure is to be considered merely as an exemplification of the principles of the invention and is not intended to limit the broad aspect of the invention to the specific embodiments illustrated.

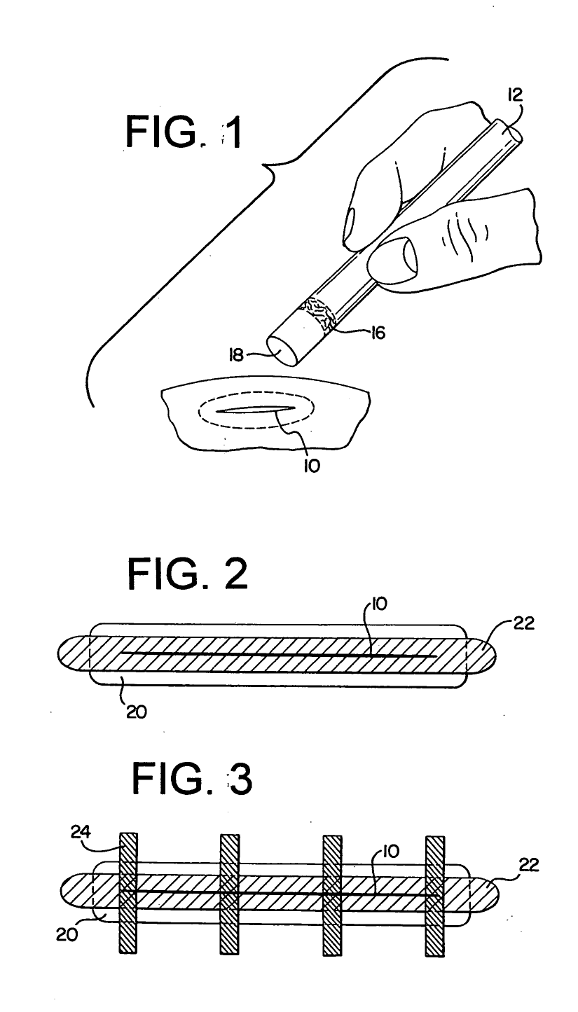

According to the present invention, with reference to FIGS. 1-3, the treatment of a wound comprises the use of the adhesive material 1-butyl cyanoacrylate 16. This topical skin adhesive is sold under the commercial name Indermil™, Histacryl Blue™, and Vetbond™ and is applied to a wound site 10 through the use of an applicator 12. A vial (not shown) inside the applicator 12 stores the unused 1-butyl cyanoacrylate 16 until application, and the material is applied to the wound site 10 via the applicator tip 18. While in the applicator 12, the unused 1-butyl cyanoacrylate 16 is in liquid form. The material will quickly set after it is applied to the wound site 10.

Under the present invention, 1-butyl cyanoacrylate 16 adhesive can be applied to wounds located both internally and externally on either a human or animal patient. More specifically, this invention is directed to high-tension wounds. The term “wound” is intended to include wounds resulting from surgical incisions, lacerations, punctures, and the like. “High-tension” wound sites are defined as areas at or near a joint, and include areas at or near an elbow or knee. It is important to provide extra tensile strength to high-tension wounds to prevent a high rate of reepithelialization that causes severe scars. In the case where the wound is located in a high tension area, the joint should preferably be tensioned to at least about a 45-degree angle before applying the adhesive in order to compensate for any stretching of the area that may occur after application

In one embodiment of the present invention, 1-butyl cyanoacrylate 16 is applied to a wound site 10 as shown in FIGS. 1-3. After the wound site 10 is defined and cleaned, the user squeezes the applicator 12, causing some 1-butyl cyanoacrylate 16 to seep through the applicator tip 18. The user then applies multiple layers of 1-butyl cyanoacrylate 16 to the wound site 10 according to the present invention.

One embodiment of the present invention is directed to both short, high-tension wounds and long, high-tension wounds. A short, high-tension wound is a wound that is about 2.5 cm (0.984 in.) or smaller. A long, high-tension wound is a wound that is greater than about 2.5 cm (0.984 in.). When treating a short, high-tension wound preferably two separate layers, a first layer 20 and a second layer 22 of adhesive material are applied to wound site 10, with none of the layers 20 and 22 extending more than about five millimeters away from the nearest edge of the wound site 10. The second layer 22 is preferably applied to the wound site 10 at least about ten to fifteen seconds after the first layer 20 has been applied, allowing the first layer 20 to properly set and polymerize. For long, high-tension wounds where a third layer 24 is required, the third layer 24 is preferably applied about ten to fifteen seconds after the second layer 22 has been applied, allowing both the first layer 20 and the second layer 22 to properly set and polymerize. Before and while applying the layers 20, 22, and 24 the user may approximate the wound edges with their fingers while wearing latex-free gloves.

In one embodiment, for short, high-tension wounds, the first layer 20 should be applied directly over the wound site 10 extending at least about 5 mm (0.197 in.) from each edge of the wound site 10. After the first layer 20 has properly set and polymerized, the second layer 22 should be applied directly over the wound site 10 extending at least about 2.5 mm (0.0984 in.) beyond both sides of the wound site 10. The second layer 22 should cover a portion of the first layer 20, and the second layer 22 should also extend at least about 2.5 mm (0.0984 in.) beyond the first layer 20 on both ends as shown in FIG. 2. It is important that at least 2.5 mm (0.0984 in.) of the extending edge of the second layer 22 covers the first layer 20. Under this arrangement, the application allows the tension, created by the layers, to be more evenly distributed across the material covering the wound site 10. This placement also aids in more evenly distributing the tension created by 1-butyl cyanoacrylate while increasing the overall strength of the dressing.

Additionally, there are other variations to the method previously described for applying 1-butyl cyanoacrylate to a long, high-tension wound. For example, the first layer 20 should be applied directly over the wound site 10 extending at least about 5 mm (0.197 in.) from each edge of the wound site 10. After the first layer has properly set and polymerized, the second layer 22 should be applied directly over the wound site 10 extending at least about 2.5 mm (0.0984 in.) beyond both sides of the wound site 10. The second layer 22 should cover a portion of the first layer 20. The second layer 22 will also extend at least about 2.5 mm (0.0984 in.) beyond the first layer 20 on both ends. After the second layer 22 has properly set and polymerized, the third layer 24 should be applied so that it covers a portion of the first layer 20, a portion of the second layer 22, and a portion of the wound site 10. The third layer 24 may be a strip that is at least about 5 mm in width. The third layer 24 should be substantially perpendicular to the first and second layers 20 and 22, as well. Where the third layer is multiple strips, as shown in FIG. 3, the strips should occur at least about every 2.5 cm (0.984 in.) over the length of the wound site 10. The third layer 24 will extend beyond each edge of the first layer 20 by at least about 5 mm (0.197 in.). The additional third layer aids in strengthening the entire wound dressing and helps distribute the tension across the dressing. The enlarged surface area created by this application decreases the surface tension on the dressing. Furthermore, by decreasing the surface tension, the rate of reepithelialization is decreased.

In addition to the 1-butyl cyanoacrylate, the adhesive may comprise additional cyanoacrylate polymers including an oxyalkene, and alkylene carbonate, and alkyl ester and an alkyl cyanoacrylate. While preferred embodiments have been shown and described, it should be understood that changes and modifications can be made therein without departing from the invention in its broader aspects. For example, it is possible that 1-butyl cyanoacrylate could be applied in slightly different locations relative to the wound site, or that a different number of layers and orientations could be used to create an effective dressing. Furthermore, it is possible that other materials with properties similar to 1-butyl cyanoacrylate, such as a compound selected from the group consisting of oxyalkene, alkylene carbonate, alkyl ester, and alkyl 1-butyl cyanoacrylate, could be used on a wound while still creating an effective wound dressing in accordance with the invention's broader aspects.

In addition to the applications described above, the present invention may also be used to treat wounds on skin having poor elasticity or poor skin turgor. An example of this type of skin includes the skin of the elderly and patients on certain medications, such as steroids. The poor skin turgor is typically less elastic than that of an otherwise healthy adult because such skin is typically thinner than that of an adult. Another example of skin having low elasticity is skin that is already stressed, swollen or otherwise distended. This type of skin is already pre-stretched and therefore is naturally less elastic than prior to the pre-stressing. A common problem associated with directly applying an adhesive to a wound surrounded by skin having poor elasticity is that of dehiscence. That is, due to the exceptional bonding and holding properties of the adhesives currently in use, the skin immediately surrounding the edge of the applied adhesive typically will tear.

An additional problem with applying adhesive directly to a wound of a pediatric patient is that the young patient often will not be able to resist the urge to pick at or peel the adhesive away from the skin. Again, due to the bonding power of the adhesive, this typically results in additional tearing or other damage to the skin surrounding the wound.

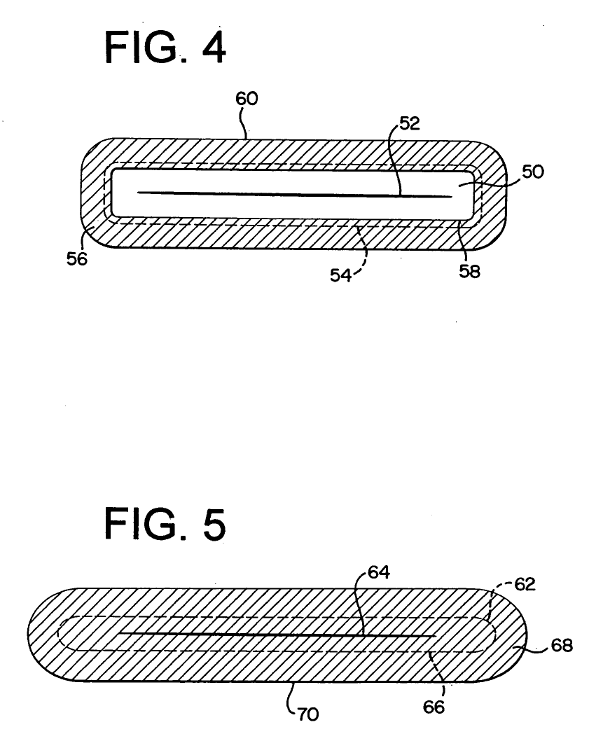

The methods of the present invention may be utilized to treat wounds in skin having poor elasticity. In one embodiment of the present invention, a first layer 50 of butyl-cyanoacrylate is applied to a wound 52 as shown in FIG. 4. A first layer edge 54 should preferably extend at all locations to a distance of approximately 5 mm (0.197 in.) from the wound 52. A second layer 56 of butyl-cyanoacrylate is then applied to the wound 52. The second layer 56 overlaps a portion of the first layer 50 and a portion of the skin surrounding the wound 52. In one embodiment, an inner edge 58 of the second layer 56 overlaps the first layer edge 54. It is preferred that the inner edge 58 of the second layer 56 overlap the first layer edge 54 by at least 1 mm (0.039 in.). An outer edge 60 of the second layer 56 will extend away from the wound 52. It is preferred that the outer edge 60 of the second layer 56 will extend to no less than about 1 cm (0.394 in.) from the wound 52.

It has been found that this arrangement of adhesive layers 50, 56 reduces the occurrence of dehiscence. Additionally, it has also been found that the second layer 56 reduces the ability of the patient to pick at or attempt to peel the adhesive from the skin.

Further in addition to the above, the present invention may also be used to treat wounds in skin subject to moderate tension, such as that found in diabetic patients, patients having a large percentage of adipose tissue, or those on long term steroid therapy.

As shown in FIG. 5, a first layer 62 of butyl-cyanoacrylate is applied to the wound 64 such that an outer edge 66 of the first layer 62 extends no less than about 5 mm (0.197 in.) from the wound 64 in all directions. A second layer 68 of butyl-cyanoacrylate is applied to the wound 64 such that it entirely covers the first layer 62 and extends beyond the outer edge 66 of the first layer 62. It can be seen that the second layer 68 covers the first layer 62 and a portion of the skin surrounding the wound. An outer edge 70 of the second layer 68 preferably extends no less than 5 mm (0.197 in.) past the outer edge 66 of the first layer 62. This arrangement of adhesive layers 62, 68 has been found to be superior in treating wounds to skin subjected to moderate tension.

While the specific embodiments and various details thereof have been illustrated and described, numerous modifications come to mind without significantly departing from the spirit of the invention and the scope of protection is only limited by the following claims:

1. A method for treating a wound on a pediatric patient or a patient having poor skin turgor, the method comprising the steps of:
preparing a wound site for treatment;
applying a first layer comprising a butyl-cyanoacrylate adhesive directly to the wound site such that a peripheral edge of the first layer extends beyond all edges of the wound site;
applying a second layer comprising a butyl-cyanoacrylate adhesive directly over the peripheral edge of the first layer and extending beyond all edges of the wound.

2. The method of claim 1, wherein the first layer extends beyond all edges of the wound site by no less than about 2 millimeters.

3. The method of claim 2, wherein the first layer extends beyond all edges of the wound site by no less than about 5 millimeters.

4. The method of claim 1, further comprising the step of polymerizing the first layer on the wound site.

5. The method of claim 4, wherein the step of polymerizing the first layer comprises the step of delaying the application of the second layer of a butyl-cyanoacrylate adhesive for a period of time.

6. The method of claim 5, wherein the period of time of the step of delaying the application of the second layer of a butyl-cyanoacrylate adhesive is within the range of from about 5 to about 20 seconds.

7. The method of claim 5, wherein the period of time is within the range of from about 5 to about 15 seconds.

8. The method of claim 5, wherein the period of time is about 10 seconds.

9. The method of claim 1, wherein the step of applying a second layer of a butyl-cyanoacrylate adhesive comprises the step of overlapping the peripheral edge of the first layer by no less than 0.5 millimeter.

10. The method of claim 9, wherein the step of applying a second layer of a butyl-cyanoacrylate adhesive comprises the step of overlapping the peripheral edge of the first layer by no less than 1.0 millimeter.

11. The method of claim 1, wherein the step of applying a second layer of a butyl-cyanoacrylate adhesive comprises the step of extending the second layer no less than 0.5 centimeters from the wound site.

12. The method of claim 11, wherein the step of applying a second layer of a butyl-cyanoacrylate adhesive comprises the step of extending the second layer no less than 1.0 centimeters from the wound site.

13. The method of claim 1, wherein the first layer further comprises a compound selected from the group consisting of oxyalkene-cyanoacrylate, alkylene carbonate-cyanoacrylate, octyl-cyanoacrylate, alkyl ester-cyanoacrylate, alkyl-cyanoacrylate, and any combination thereof.

14. The method of claim 1, wherein the second layer further comprises a compound selected from the group consisting of oxyalkene-cyanoacrylate, alkylene carbonate-cyanoacrylate, octyl-cyanoacrylate, alkyl ester-cyanoacrylate, alkyl-cyanoacrylate, and any combination thereof.

15. A dressing for a wound site on a pediatric patient, said dressing consisting of:
a first layer comprising a butyl-cyanoacrylate adhesive applied directly to the wound site and extending a distance beyond the wound site to form a peripheral edge;
a second layer comprising a butyl-cyanoacrylate adhesive applied directly to the first layer and overlapping the peripheral edge of the first layer a distance.

16. The dressing of claim 15, wherein the distance the first layer extends beyond the wound site is within a range of from about 2 to about 15 millimeters.

17. The dressing of claim 16, wherein the distance the first layer extends beyond the wound site is within a range of from about 5 to about 12 millimeters.

18. The dressing of claim 17, wherein the distance the first layer extends beyond the wound site is about 5 millimeters.

19. The dressing of claim 15, wherein the distance the second layer overlaps the peripheral edge is not less than about 0.5 millimeters.

20. The dressing of claim 19, wherein the distance the second layer overlaps the peripheral edge is not less than about 1.0 millimeters.

21. The dressing of claim 15, wherein the second layer is applied to overlap the peripheral edge of the first layer such that peeling of the first layer is minimized.

22. The dressing of claim 15, wherein the first layer further comprises a compound selected from the group consisting of oxyalkene-cyanoacrylate, alkylene carbonate-cyanoacrylate, octyl-cyanoacrylate, alkyl ester-cyanoacrylate, alkyl-cyanoacrylate, and any combination thereof.

23. The dressing of claim 15, wherein the second layer further comprises a compound selected from the group consisting of oxyalkene-cyanoacrylate, alkylene carbonate-cyanoacrylate, octyl-cyanoacrylate, alkyl ester-cyanoacrylate, alkyl-cyanoacrylate, and any combination thereof.
